(19) United States
(12) Patent Application Publication
(10) Pub. No.: US 2004/0162476 A1
(43) Pub. Date: Aug. 19, 2004

(54) Positioning device for x-ray examinations

(76) Inventor: Lars Selander, Huddinge (SE)

Correspondence Address: YOUNG & THOMPSON, 745 SOUTH 23RD STREET 2ND FLOOR, ARLINGTON, VA, 22202

(21) Appl. No.: 10/479,413

(22) PCT Filed: Jun. 4, 2002
(86) PCT No.: PCT/SE02/01061

(30) Foreign Application Priority Data
Jun. 6, 2001 (SE) 0101964-5

(51) Int. Cl. A61B 5/05

(52) U.S. Cl. 600/407

(57) ABSTRACT

The present invention relates to a positioning device for x-ray examinations, said device comprising a base (1) and a supporting unit (11) supported by the base (1), said supporting unit (11) having means (13) for carrying the seat of a patient (20) and supporting means (16, 17) for cooperation with the parts of the patient (20) that are located above the seat when the patient is seated. It is significant for the positioning device according to the present invention that the base (1) and the supporting unit (11) are mutually connected via a hinge (10), and that the device comprises means (19) to effect a tilting, via the hinge (10), of the supporting unit (11) relative to the base (1).

### TECHNICAL FIELD OF THE INVENTION

The present invention relates to a positioning device for x-ray examinations, said device comprising a base and a supporting unit supported by the base, said supporting unit having means for carrying the seat of a patient and supporting means for cooperation with the parts of the patient that are located above the seat.

1. Prior Art

In connection with certain x-ray examinations, e.g. x-ray of the chest where liquid being the question at issue, the patient is placed on its side and a horizontal x-ray photograph is taken of the downward hanging lung where possible liquid in the pleural sack is spread and constitute a liquid level. In connection therewith the are considerable problems to locate the patient in correct position on a horizontal bed that is provided with a recess. Normally the patient must be supported by pillows or other supports.

From SE-B-460 011 it is previously known a chair for locating a patient in desired positions in connection with x-ray examinations, especially of the lungs and the pleural sack. This chair functions in such a way that initially a cradle is located in desired position, said cradle being supported by the chair. In order to achieve this the chair is tilted around a horizontal axis of rotation, said axis going through the patient. Then the chair is elevated in a second phase up to a desired level and the final position is reached. In this position the x-ray photographs are taken. Thus an operation in two steps is required in order to achieve the final position of the patient.

2. Objects and Features of the Invention

A primary object of the present invention is to disclose a positioning device of the type defined above, the patient being transferred in principle in one continuous step from the initially seated position to the final examination position where x-ray photographs are taken.

A further object of the present invention is to have the positioning device structurally simple and in principle constituting only two parts that are mutually connected by means of one hinge.

Still an object of the present invention is to give the different parts of the patient a proper support during the entire tilting motion that a supporting unit of the positioning device performs when carrying out an x-ray examination.

At least the primary object of the present invention is realised by means of a positioning device that has been given the features of the appending independent claim. Preferred embodiments of the invention are defined in the independent claims.

### BRIEF DESCRIPTION OF THE DAWINGS

Below an embodiment of the invention will be described, reference be made to the accompanying drawings, where:

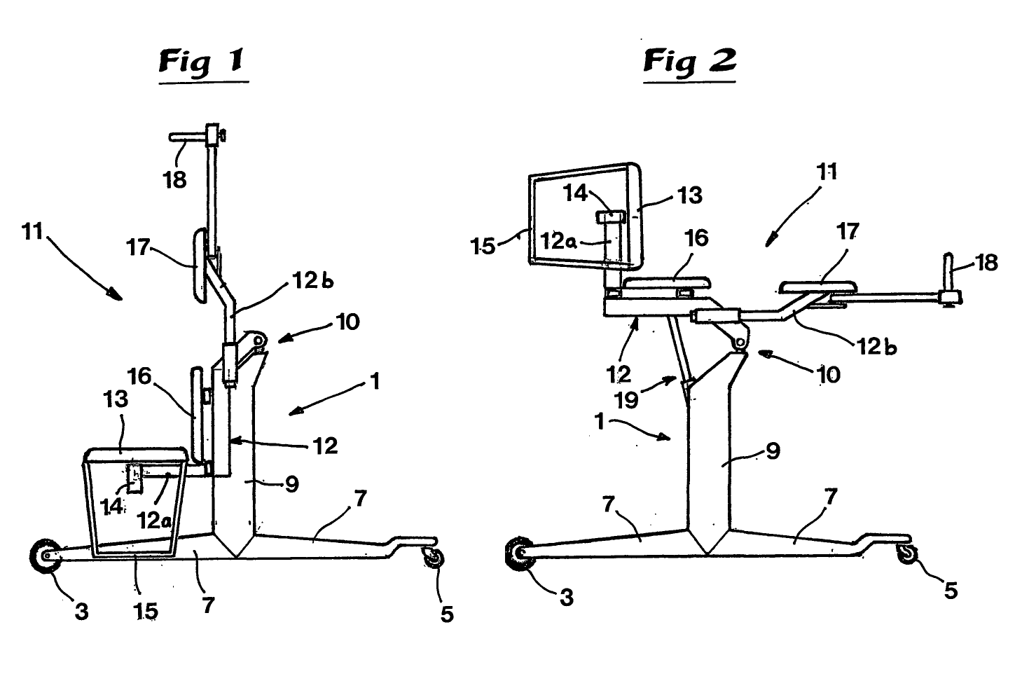

FIG. 1 shows a side view of the positioning device according to the present invention, said device being in initial position;

FIG. 2 shows a side view of the positioning device according to the present invention, said device being in examination position, i.e. in position for taking x-ray photographs;

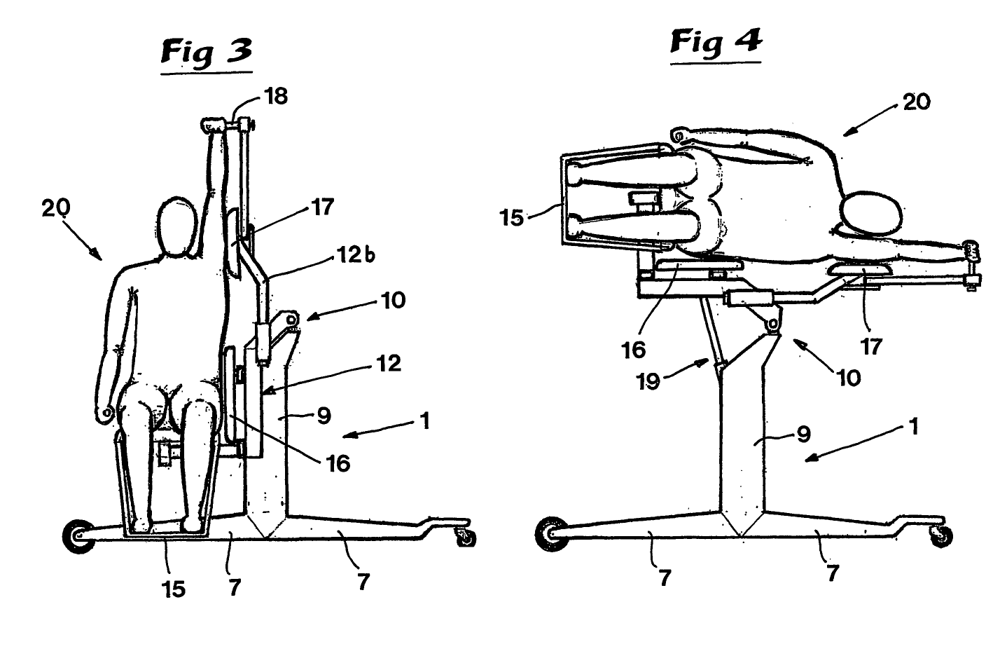

FIG. 3 shows a side view of the positioning device according to the present invention, said device being in initial position and a patient has been seated on a seat cushion of the device;

FIG. 4 shows a side view of the positioning device according to the present invention, the device being in examination position, i.e. a position for taking X-ray photographs and the patient has been transferred to a lying position.

### DETAILED DESCRIPTION OF A PREFERRED EMBODIMENT OF THE INVENTION

In FIGS. 1 and 2 the positioning device according to the present invention is shown, said device comprising a base 1 that in side view generally is T-shaped. The base 1 is supported by two first wheels 3 and two second wheels 5, the second wheels 5 preferably being so-called swivel castors, said wheels making the base 1 more easy to handle in small spaces a stop the base 1 comprises four first portions 7, only two of said portions being shown in FIGS. 1 and 2. The first portions 7 have an extension along the ground that the base 1 is resting upon. The base 1 also has a second portion 9, generally in the shape of an erected column that extends substantially perpendicular to the first portions 7 that extend radially, in star-shape, from the second portion 9. At the free ends of the first portions 7 the first and second wheels 3 and 5 respectively are attached.

At the free end of the second portion 9 a hinge 10 is attached, said hinge 10 connecting the base 1 with a supporting unit 11 that comprises a generally L-shaped frame 12 that in its turn comprises a shank 12a and a stem 12b. Generally the frame 12 constitutes welded profiles, preferably square profiles. On the shank 12a of the frame 12 a seat cushion 13 is attached, said seat cushion 13 having an extension parallel to the extension of the shank 12a. The seat cushion is connected to the shank 12a via a rotatable journalling means 14 that allows the seat cushion 13 to rotate in its main plane depending on which side of the patient that is to be examined. In the shown embodiment the seat cushion 13 is equipped with a foot rest 15 that follows when the seat cushion 13 is rotated.

First and second supporting pads 16 and 17 respectively are provided on the stem 12b of the frame 12, said supporting pads 16, 17 being arranged that a certain distance from each other. Preferably the supporting pads 16, 17 are located in a common plane and are adjustable relative to the seating cushion 13 in a direction along the longitudinal direction of the stem 12b. The mutual distance between the supporting pads 16 and 17 is also adjustable.

In the area of the upper free end of the stem 12b a handle 18 is provided, said handle 18 extending transverse to the longitudinal direction of the stem 12b.

In order to effect tilting of the supporting unit 11 relative to the base 1, via the hinge 10, a pressure medium cylinder 19 is provided, said cylinder 19 having it ends attached to the second, erected portion 9 of the base 1 and to the stem 12b of the supporting unit 11 respectively. This pressure medium cylinder 19 may either be activated by of motor or manually, e.g. via a foot operated pedal. In connection with a prolongation of the pressure a medium cylinder 19 the supporting unit 11 will be transferred from the position shown in FIG. 1 to the position shown in FIG. 2.

The use of the positioning device according to the present invention is illustrated in FIGS. 3 and 4. In FIG. 3 the positioning device according to the present invention is in its initial position, the L-shaped frame 12 of the supporting unit 11 assuming a position where the stem 12b is essentially vertical, i.e. it extends essentially perpendicular to the first portion 7 of the base 1. A patient 20 has been seated on the seating cushion 13, said patient 20 resting his/her feet against the foot rest 15. The patient 20 has also grabbed around the handle 18 with the hand of the arm that is closest to the stem 12b of the frame 12. Relevant parts, e.g. thigh, hip and arm, of the patient 20 rests against or are in the vicinity of a the supporting pads 16 and 17. Since the supporting pads 16, 17 are adjustable as regards their positions relative to the seating cushion 13 individual adaption of the positions of the supporting pads 16, 17 may be effected in order to bring them opposite to the desired parts of the patient 20. This is normally the initial position of the patient 20.

In order to transfer the patient 20 to an examining position where x-ray photographs can be taken the supporting unit 11 is tilted, via the hinge 10, relative to be base 1. In order to effect this tilting the pressure medium cylinder 19 is activated. When the pressure medium cylinder 19 is prolongated the supporting unit 11/the patient 20 will be transferred from the initial position according to FIG. 3 to the examining position according to FIG. 4. Further adjustment of the position of the patient 20 may then take place since the base 1 is mobile relative to the ground. As is evident from a comparison between FIG. 3 and FIG. 4 the patient 20 assumes in principle the same position relative to the supporting unit 11 also subsequent to the tilting of the supporting unit 11 relative to the base 1. In this connection it should however be pointed out that subsequent to the transfer of the supporting unit 11 to the examining position according to a FIG. 4 relevant portions of the patient 20 will rest against the supporting pads 16 and 17.

Since the hinge 10, that constitutes centre of rotation of the tilting motion of the supporting unit 11 relative to the base 1, is located lateral of the patient 20 the elevation of the patient 20 will be higher compared to the case when the centre of rotation passes through the patient 20. The transfer of the initial position according to FIG. 3 to the examining position according to FIG. 4 is effected solely by tilting the supporting unit 11 around the hinge 10.

When the x-ray examination is finished the supporting unit 11 is transferred from the position according to FIG. 4 to the position according to FIG. 3. The patient 20 may now in a simple way release the grip around the handle 18 and descend from the seating cushion 13.

### FEASIBLE MODIFICATIONS OF THE INVENTION

In order to further improve the feeling of security, especially when the supporting unit 11 is transferred to or is located in the examining position, it is possible within the scope of the present invention to equip the supporting unit 11 with some kind of fastening device, e.g. of the type safety belt.

An alternative way to improve the feeling of security of the patient is to equip the seating cushion 13 with a second handle that is located at the same level has the seating cushion 13 and provided on the side of the seating cushion 13 that faces from the lower supporting pad 16. Thereby, the patient may grab handles with both arms when there is a transfer to an examining position.

In the embodiment shown above the tilting of the supporting unit 11 relative to the base 1 is effected by means of a pressure medium cylinder 19. However, within the scope of the present invention it is feasible to have other types of effecting means to achieve said tilting of the supporting unit 11. In exemplifying and non-restricting purpose chain transmission and/or toothed transmission gear.

1. Positioning device for x-ray examinations, said device comprising a base (1) and a supporting unit (11) supported by the base (1), said supporting unit (11) having means (13) for carrying the seat of a patient (20), that the base (1) and the supporting unit (11) are mutually connected via a hinge (10), that the device comprises means (19) to effect that tilting, via the hinge (10), of the supporting unit (11) relative to the base (1), that the supporting unit (11) comprises a generally L-shaped frame (12), that the seating cushion (13) is provided on the shank (12a) of the L, and that supporting means (16, 17) are provided on the stem (12b) of the L to cooperate with the parts of the patient (20) that are located above the seat when the patient is seated, characterized in that in examining position of the supporting unit (11), i.e. when the patient (20) generally assumes a horizontal position, the hinge (end) is located between the base (1) and the supporting unit (11).

2. Device according to claim 1, characterized in that the means to effect tilting of the supporting unit (11) relative to the base (1) constitutes a pressure medium cylinder (19).

3. Device according to claim 1 or 2, characterized in that the supporting means on the stem of the L constitute supporting pads (16, 17), and that the supporting pads (16, 17) have an extension essentially perpendicular to the extension of the seating cushion (13).

4. Device according to any of the previous claims, characterized in that the seating cushion (13) is provided with a footrest (15).

5. Device according to any of the previous claims, characterized in that the supporting unit (11), in the area of the end facing from the seating cushion (13), is equipped with a handle (18).

6. Device according to claim 3, characterized in that the positions of the supporting pads (16, 17) may be adjusted along the stem (12b) of the L.

7. Device according to any of the previous claims, characterized in that be seating cushion (13) is rotatable in its main plane.

8. Device according to any of the previous claims, characterized in that the base (1) on one hand comprises a number of first portions (7) that have an extension along the ground that the base (1) rests upon and on the other hand a second portion (9), generally in the shape of an erected column that extends essentially perpendicular to the first portions (7) that extends radially, in star shape, from the second portion (9).

9. Device according to any of the previous claims, characterized in that the base (1) is equipped with wheels (3, 5).
